(19) United States
(12) Patent Application Publication
(10) Pub. No.: US 2020/0165197 A1
(43) Pub. Date: May 28, 2020

(54) SULFONAMIDE MACROMOLECULES USEFUL AS SINGLE-ION CONDUCTING POLYMER ELECTROLYTE

(71) Applicant: COMMISSARIAT A L'ENERGIE ATOMIQUE ET AUX ENERGIES ALTERNATIVES, Paris (FR)

(72) Inventors: Philippe OVERTON, Grenoble (FR); Lionel PICARD, Seyssinet-Pariset (FR); Patrice RANNOU, Eybens (FR)

(21) Appl. No.: 16/628,628

(22) PCT Filed: Jul. 4, 2018
(86) PCT No.: PCT/EP2018/068135

§ 371 Date: Jan. 3, 2020

(30) Foreign Application Priority Data
Jul. 4, 2017 (FR) 1756291

(51) Int. Cl. C07C 311/08; H01M 10/0565; H01M 10/052; H01M 8/1018; H01M 6/18; C08L 25/18; H01M 8/18

(52) U.S. Cl. CPC C07C 311/08; H01M 10/0565; H01M 10/052; H01M 2300/0082; H01M 6/181; C08L 25/18; H01M 8/188; H01M 8/1018

(57) ABSTRACT

The present invention relates to asymmetric sulfonamide compounds comprising at least: one polycyclic group, Ar, formed of two to six rings, at least one of which is aromatic, a linear or branched, saturated or unsaturated aliphatic chain, said chain possibly being interrupted by one or more heteroatoms, said group Ar and said aliphatic chain being covalently bonded via a spacer represented by a sulfonamide unit —SO₂—NH— or its anionic form —SO₂—N⁻-; and, optionally a counter-cation of the anionic form of the sulfonamide unit, chosen among the alkali metals and the proton H⁺. These compounds are of particular interest as single-ion conducting polymer electrolyte.

The present invention relates to the field of single-ion conducting polymer electrolytes and aims in particular to propose novel asymmetric polymer salts for the transport of cations, such as lithium, in electrochemical systems.

The main electrochemical systems use lithium cation as a transport ionic species. For example, in the case of a lithium battery, the lithium cation extracted from the cathode during battery charging is deposited on the anode and, conversely, is extracted from the anode and inserted into the cathode during discharge. It is transported by an ion conductive electrolyte.

The nature of the ion conductive electrolyte used is essential for the performance of the electrochemical system. The ionic conductivity of the electrolyte determines, among other things, the efficiency of the electrochemical system since it affects the mobility of ions between the positive and negative electrodes.

Other parameters are also involved in the choice of electrolyte used. These include its thermal, chemical or electrochemical stability within the electrochemical system, as well as economic, safety and environmental criteria.

Generally, the electrolytes in electrochemical systems are in liquid, gel or solid form.

The present invention focuses on electrolytes in solid form, and more particularly on solid polymer electrolytes, which are safer and do not necessarily require the use of a separator.

An illustrative example of these polymer electrolytes is poly(oxyethylene), or POE, in which, conventionally, an alkali or alkaline earth metal salt is dissolved depending on the chemistry of the electrodes. However, these electrolytes have limited ionic conductivity performance due to the so-called “assisted” cation transport mechanism and require a high operating temperature (60° C. to 80° C.).

Various studies have therefore been carried out to improve the ion conductive performance of polymer electrolytes.

For example, a mixture of POE and of polystyrene with sulfonyl(trifluoromethylsulfonyl)imide groups has been proposed to form an electrolyte membrane (Meziane et al. Electrochimica Acta, 2011, 57, 14-19). However, these polymer electrolytes have insufficient ionic conductivity, of the order of 9.5-10 S/cm at 70° C.

The incorporation of a lithium bis(trifluoromethane)sulfonimide (LiTFSI) salt into the repeating unit, together with styrene, to form poly(styrene trifluoromethanesulfonylimide of lithium) P(STFSILi), has also been considered for lithium electrolyte systems. At the end of the polymerization, the poly(electrolyte) has a BAB block copolymer architecture with a central PEO block “A”. The maximum conductivity, of the order of 10⁻⁵ S/cm, is obtained at 60° C. with a polymer containing 78 wt. % POE. (R Bouchet et al., Nature Materials (2013), 12, 452). The incorporation of a functional group at the end of a block copolymer of this type has proven to be beneficial for its conductivity (G. Lo et al., ACS Macro Lett. (2013), 2, 990 and H. Jung et al., Macromolecules (2017), 50, 3224).

For obvious reasons, improving electrolyte performance is an ongoing objective.

Therefore, there remains a need for an electrolyte with high ionic conductivity and preferably a cation transport number as close to unity as possible.

There also remains a need for an electrolyte with enhanced electrochemical stability.

The present invention aims precisely to propose novel compounds useful as ion-cation- or proton-conducting electrolytes because they have enhanced ionic or protonic conductivity and enhanced electrochemical stability.

More particularly, it concerns, according to a first aspect, an asymmetric sulfonamide compound useful as, among other things, a single-ion conducting polymer electrolyte, comprising at least:
a polycyclic group, Ar, consisting of two to six rings, at least one of which is aromatic, said rings each independently having from 4 to 6 members, said polycyclic group being able to include up to 18 heteroatoms, in particular selected from the group consisting of S, N and O;
a linear or branched, saturated or unsaturated aliphatic chain, said chain being optionally interrupted by one or more heteroatoms, by one or more metalloids, optionally substituted by one or more fluorine atoms, and/or containing one or more groups selected from the group consisting of hydroxyl, —NH₂ and oxo groups;
said group Ar and said aliphatic chain being covalently linked via a spacer represented by a sulfonamide unit —SO₂—NH—, or its anionic form —SO₂—N⁻—; and, if need be
a counter cation for the anionic form of the sulfonamide unit, selected from the alkali metals and the proton H⁺ or H₃O⁺.

In particular, the compounds according to the invention are in the form of a salt having, as spacer, the anionic form of sulfonamide —SO₂—N⁻—, and a counter cation, preferably alkaline, and in particular a lithium atom.

In particular, the sulfonamide unit is linked to said polycyclic group via its nitrogen atom and to said aliphatic chain via its sulfur atom.

More particularly, the asymmetric sulfonamide compounds according to the invention comprise at least:
a polycyclic group, Ar, consisting of two to six rings, at least one of which is aromatic, said rings each independently having from 4 to 6 members, said polycyclic group being able to include up to 18 heteroatoms, in particular selected from the group consisting of S, N and O;
a linear or branched, saturated or unsaturated aliphatic chain, said chain being optionally interrupted by one or more heteroatoms, by one or more metalloids, optionally substituted by one or more fluorine atoms, and/or containing one or more groups selected from the group consisting of hydroxyl, —NH₂ and oxo groups, said aliphatic chain comprising a linear chain of at least six covalent bonds;
said group Ar and said aliphatic chain being covalently linked via a spacer represented by a sulfonamide unit —SO₂—NH— or its anionic form —SO₂—N⁻—, said sulfonamide being linked to said polycyclic group Ar via its nitrogen atom and to said aliphatic chain via its sulfur atom; and, if need be
a counter cation for the anionic form of the sulfonamide unit, selected from the alkali metals and the proton H⁺ or H₃O⁺.

In particular, the covalent bond between the nitrogen atom and said polycyclic group is established via an aromatic structure forming all or part of said polycyclic group.

In particular, the polycyclic group Ar is in a condensed aromatic form.

Admittedly, sulfonamides have already been considered for the development of (co)-solvents for lithium-ion batteries. However, the architecture of the corresponding compounds is very different. The compounds essentially have a linking of the sulfur atom of the sulfonamide to short fluorinated alkyl chains and the nitrogen of this sulfonamide to simple hydrocarbon functions (D Bresser et al. EP 3 050 872).

Sulfonamides bound to an aromatic species are rare in the published literature. To the knowledge of inventors, only the publication H. Ohno et al. (Solid State Ionics (1999), 124, 323) describes compounds whose sulfonamide unit is bound via its nitrogen atom to phenyl. This asymmetric salt can also be connected via the sulfur atom of the sulfonamide anion to a PEO polymer (550 g/mol). Consequently, the compounds described in this document are devoid of the polycyclic unit considered according to the invention.

However, the compounds according to the invention, when used as electrolytes, are of particular interest in view of their novel feature of having such a polycyclic group.

As can be seen from the above, the nitrogen atom of the anionic sulfonamide unit is covalently bound to an aromatic group (Ar). In turn, the sulfur atom of the sulfonamide unit is bound to an oligomeric or polymeric group. It is the deprotonation of the sulfonamide unit that generates the Lewis base. However, advantageously, the Lewis base character of sulfonamide is increased by the aromatic structure, to which it is bound via its nitrogen atom. Indeed, the negative charge of the Lewis base is delocalized on the oxygen atoms of the sulfonamide, in relation to the z electrons delocalized at the aromatic structure. Thus, the aromatic structure increases the physicochemical characteristics of the asymmetric salt in two ways.

First, it contributes to the electronic stabilization of salt by delocalizing its anionic charge. The aromatic structure, located nearby because it is bound to the nitrogen atom of the sulfonamide, reinforces the anionic electron density and this delocalization thus improves the dissociation of the cation, generally lithium, from the anionic component of the compound according to the invention.

Furthermore, the aromatic structure contributes by its size, as directional structuring agent, to the structural organization of the polymer in the solid state. A monocyclic aromatic unit, which is smaller in size than a phenyl radical, is less able to direct the structural organization of the salt than a larger polycyclic unit. The known π-π stacking interactions (van der Waals) of the proximal aromatic rings have a significant effect in directing the organization of the structure, in combination with the steric volume of the polycyclic aromatic structure. A functionalized naphthalene group is known to self-assemble via π-π stacking (van der Waals interaction) and the dimensions of a larger aromatic ring, particularly in relation to benzene, introduce a steric dimension favorable to this self-assembly. The inventors thus found that the size and position of the aromatic group Ar in the macromolecular architecture of the compounds according to the invention are decisive parameters for the effectiveness of the electrolytes derived therefrom.

Advantageously, the ionic conductivity of an electrolyte according to the invention is based on a “direct” conduction mechanism by “hopping” the C^{x+} cations of the anionic sulfonamide group located on one polycyclic group Ar to the other.

According to another aspect, the present invention relates to the use of the anionic form of an asymmetric sulfonamide according to the invention as electrolyte.

According to yet another of its aspects, the present invention relates to an electrolyte formed in whole or in part by the anionic form of an asymmetric sulfonamide according to the invention.

In particular, the anionic form considered is an alkaline salt, and in particular the lithium salt.

The macromolecules according to the invention can be used as electrolytes in many electrochemical systems, such as generators, for example lithium batteries, and electrochemical conversion systems, for example proton exchange membrane fuel cells (PEMFCs).

Other features, variants and advantages of the molecules and electrolytes according to the invention, their preparation and their implementation will emerge better from the description, examples and figures that will follow, given by way of non-limiting illustration of the invention.

Hereinafter, the terms “between . . . and . . . ”, “from . . . to . . . ” and “varying from . . . to . . . ” are equivalent and mean that the bounds are included, unless otherwise stated.

Unless otherwise specified, the expression “including/comprising one” should be understood as “including/comprising at least one”.

Sulfonamide Compounds of the Invention

As stated above, the asymmetric sulfonamide compounds according to the invention comprise at least:
a polycyclic group, Ar, consisting of two to six rings, at least one of which is aromatic, said rings each independently having from 4 to 6 members, said polycyclic group being able to include up to 18 heteroatoms, in particular selected from the group consisting of S, N and O;
a linear or branched, saturated or unsaturated aliphatic chain, said chain being optionally interrupted by one or more heteroatoms, by one or more metalloids, in particular containing a silicon atom, optionally substituted by one or more fluorine atoms, and/or containing one or more groups selected from the group consisting of hydroxyl, —NH₂ and oxo groups, said aliphatic chain comprising in particular a linear sequence of at least six covalent bonds;
said group Ar and said aliphatic chain being covalently linked via a spacer represented by a sulfonamide unit —SO₂—NH—, or its anionic form —SO₂—N⁻—; said sulfonamide being in particular linked to said polycyclic group Ar via its nitrogen atom and to said aliphatic chain via its sulfur atom; and, if need be
a counter cation selected from the alkali metals and the proton H⁺ or H₃O⁺.

Polycyclic Group

In the context of the invention, “polycyclic group” means a group having two or more nuclei (rings), condensed (ortho-condensed or ortho- and peri-condensed) to each other, i.e. having, in pairs, at least two common carbons.

In particular, a polycyclic group according to the invention consists of two to six rings, the rings each independently having from 4 to 6 members. The polycyclic group is said to be aromatic insofar as it comprises at least one aromatic ring which is involved in particular in the covalent bond with the nitrogen atom of the sulfonamide unit.

Thus, the polycyclic group can be partly aromatic or fully aromatic.

The polycyclic group may include one or more heteroatoms. This is referred to as a “polyheterocyclic group”.

The expression “4- to 6-membered (hetero)cycle or not” means a 4-, 5- or 6-membered unsaturated, partially saturated or saturated cyclic group, optionally including one or more heteroatoms, in particular selected from the group consisting of oxygen, sulfur and nitrogen.

In particular, the polycyclic group referred to as Ar is an aromatic polycyclic group consisting of 2 to 6 fused 6-membered aromatic rings, preferably 2 to 6 fused benzene rings, advantageously 2 to 4 fused benzene rings.

More particularly, Ar may have one of the following polycyclic skeletons or derive from one of the following polycyclic skeletons:

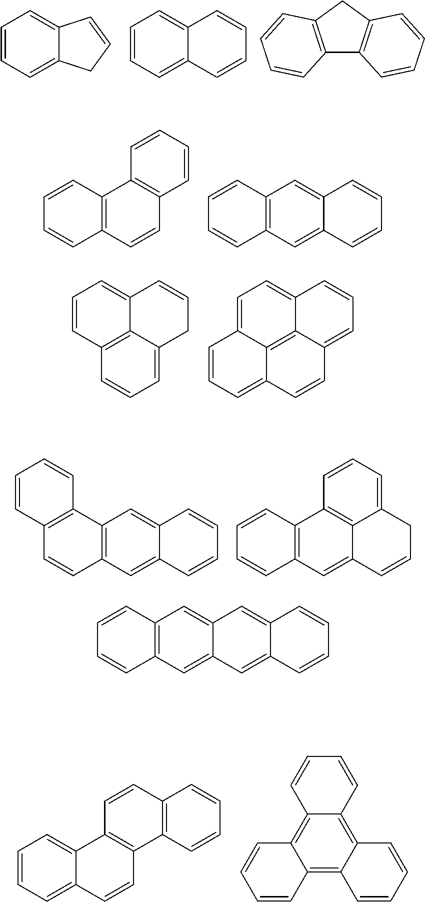

As stated above, it is understood that the group Ar may be a polyheterocyclic group, i.e. one of the skeletons presented above but in which one or more carbon atoms are replaced by one or more heteroatoms, in particular selected from the group consisting of S, N and O.

According to a particular embodiment, Ar is a radical derived from an aromatic bicyclic skeleton, and more particularly a naphthalene radical or so-called naphthyl radical.

Aliphatic Chain

In the sense of the invention, a linear or branched, saturated or unsaturated aliphatic chain may optionally have an oligomeric, homopolymeric or copolymeric structure.

In particular, a polymer can be characterized by a number of repeat units (DPn). In particular, it may have a DPn greater than 5.

According to an embodiment, the aliphatic chain includes a linear sequence of at least 6 covalent bonds.

According to a particular embodiment, it may consist of a single segment or a linear sequence of at least two chain segments, in particular two or three chain segments of a different chemical nature.

Preferably, it consists of a linear alkyl chain containing 6 to 18 carbon atoms, preferably 6 to 12 carbon atoms, optionally fluorinated, semifluorinated (i.e. with an alternation of methylene and perfluoromethylene units), or even perfluorinated, and/or optionally substituted by at least one or more hydroxyl group(s), and optionally intersected by one or more oxygen atom(s).

In particular, the aliphatic chain is an octyl radical.

In particular, the aliphatic chain has a chain of at least 5 or more ethylene oxide (EO) units.

Sulfonamide Unit

In the asymmetric sulfonamide compounds according to the invention, the sulfonamide unit may be in the form of —SO₂—NH— or —SO₂—N—N⁻—.

Preferably, it is the anionic form —SO₂—N— and the compound according to the invention further includes a cation, selected from the proton H⁺ or H₃O⁺ (hydrated proton) and alkali metals. When the cation is selected from the group of alkali metals, the compound according to the invention is then described as a salt.

According to a variant embodiment, the nitrogen atom of the sulfonamide anionic unit is covalently bound to a carbon atom or a heteroatom of the polycyclic group Ar.

As detailed hereinbelow, such macromolecules can be advantageously used as electrolyte in a lithium battery.

Alkaline Metals

In the sense of the invention, the term “alkali metals” refers to the chemical elements in the first column of the periodic table of elements, and more particularly selected from the group consisting of lithium, sodium, potassium, rubidium, and cesium.

Preferably, the alkali metal is lithium, sodium or potassium, and more preferably lithium.

According to another variant embodiment of the invention, the counter cation represents the proton H⁺ or H₃O⁺, and the sulfur atom is covalently bound to a carbon atom or a heteroatom of the polycyclic group Ar. Such macromolecules can be advantageously used as electrolyte in a proton exchange membrane fuel cell (PEMFC), a low-temperature electrolyzer, or a redox flow battery.

According to a particular embodiment, the molecules according to the invention have the following formula (I):

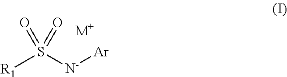

in which
Ar is a fully aromatic polycyclic group as defined above and preferably represents a naphthyl radical,
R₁ represents an aliphatic chain as defined above and
M⁺ is a counter cation selected from the group of alkali metals and in particular is Li⁺.

In particular, R₁ is a linear alkyl chain, which comprises from 4 to 18 carbon atoms, preferably from 6 to 16 carbon atoms, optionally substituted by one or more hydroxyl group(s), and/or optionally intersected by one or more oxygen atom(s).

As stated above, the linear alkyl chain can have a sequence of at least 5 or more EO units.

As representative and non-limiting sulfonamide compounds according to the invention, particular mention may be made of the following:

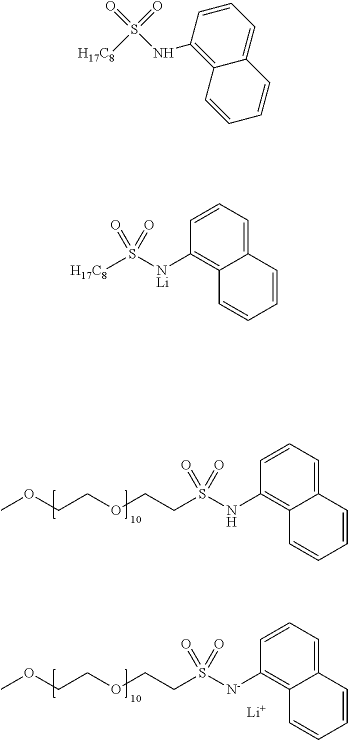

Preparation of the Compounds of the Invention

Macromolecules according to invention can be obtained according to synthesis pathways that are clearly within the competence of the skilled person.

In particular, some can be prepared by using nucleophilic substitution or addition methods known to the skilled person, as detailed below. This type of reaction involves a precursor of the polycyclic group, Ar, and a precursor of the aliphatic chain.

According to an embodiment, the Ar precursor carries a nucleophilic group, for example amine, hydroxyl or thiol, and preferably amine, and the aliphatic chain precursor carries a sulfonyl chloride electrophilic group.

The prior formation of each of the precursors may also be considered.

By way of illustration of the methods that the skilled person can use to achieve the molecules of the invention, a method for preparing the molecules of the invention of formula (I) is described below and also in the following examples.

Of course, it is up to the skilled person to adjust the synthesis conditions to obtain the macromolecules according to the invention.

Use of Sulfonamide Compounds According to the Invention as Electrolyte

According to another aspect, the present invention relates to an electrolyte comprising at least one or even consisting of an asymmetric sulfonamide salt as defined above.

The electrolyte according to the invention has good ionic conductivity properties.

Preferably, an electrolyte of the invention has an ionic conductivity at 20° C. of 10⁻⁸ S/cm or more, in particular between 10⁻⁷ S/cm and 10⁻³ S/cm and an ionic conductivity at 150° C. of 10⁻⁵ S/cm or more.

Ionic conductivity can be measured by electrochemical impedance spectroscopy in voltage or current, according to a method known to the skilled person.

The electrolyte according to the invention can be used in an electrochemical system, for example a lithium battery.

The present invention thus concerns, according to yet another of its aspects, an electrochemical system comprising an electrolyte according to the invention.

In the electrochemical system of the invention, the electrolyte is preferably impregnated on a porous separator as described above.

The electrochemical system can be a generator, converter or electrochemical storage system.

In particular, it may be a fuel cell, for example a proton exchange membrane fuel cell (PEMFC) or redox flow battery; a primary or secondary battery, for example a lithium, sodium or potassium battery; a lithium-air or lithium-sulfur battery.

According to a particular embodiment, the electrolyte is used in a battery, in particular a lithium battery.

The invention will now be described using the following examples and figures, submitted for illustrative and non-limiting purposes.

FIG. 1: Cyclic voltammetry (CV) diagram at 10 mV/s of the compound IA (example 1) in reduction mode (−0.12 V to +0.90 V vs. Ag/Ag⁺).

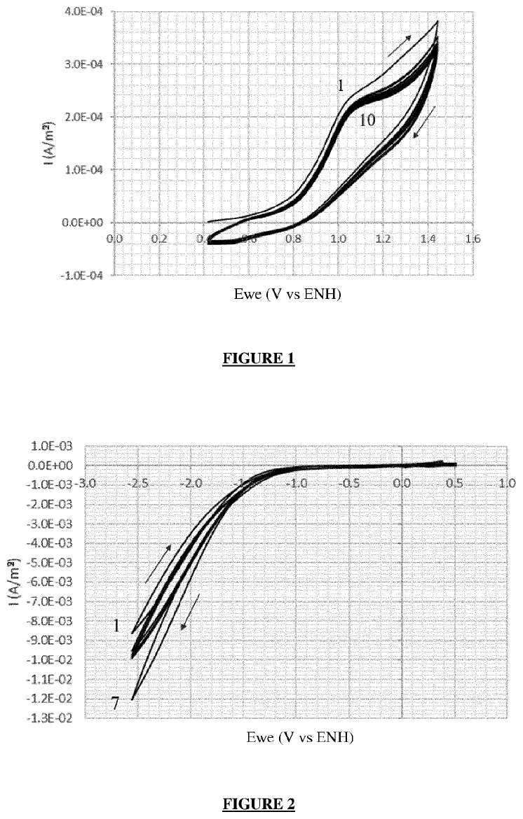

FIG. 2: Cyclic voltammetry (CV) diagram at 10 mV/s of the compound IA (example 1) (in oxidation mode (0.00 V to −3.10 V vs. Ag/Ag⁺).

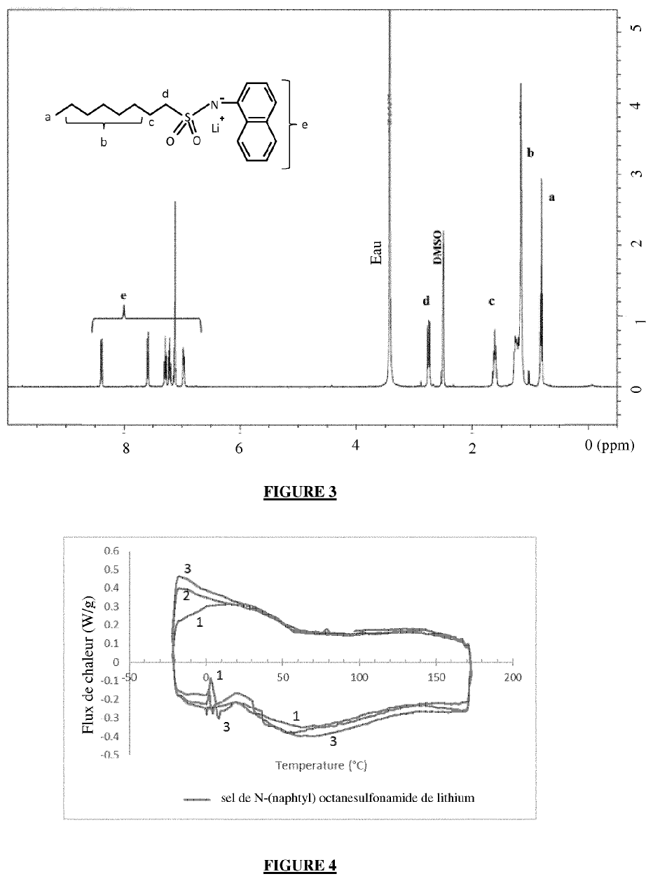

FIG. 3: 1H NMR spectrum (DMSO-d6, 400 MHz) of the compound IB

FIG. 4: Calorimetric analysis of the compound IB by DSC.

### EXAMPLE 1

Synthesis of the Compounds IA and IB According to the Invention

Step a)

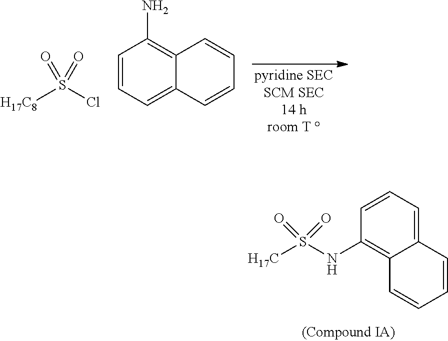

An oven-dried round-bottom flask is loaded with 1-aminonaphthalene (1-AN, 3.70 g, 25.9 mmol, 1.10 equivalent) and pyridine (4 mL). To the slightly purple and stirred solution is added 1-octanesulfonyl chloride (1-OSC, 5 g, 23.5 mmol) in DCM (1 mL), dropwise at 0° C., for 15 minutes. The reaction is exothermic. When OSC is added, the solution first forms a light, lime green color, then becomes light and shiny magenta. The solution darkens over time. After stirring for 18 hours, the solution is acidified with HCl (2 M, 12 mL), inducing phase separation. The organic phase is diluted with DCM (20 mL), separated and then extracted again with HCl (2 M, 12 mL). The aqueous phase is extracted again with DCM (2×10 mL). The organic phases are combined, extracted with water and brine, then dried over MgSO₄. The solvent is removed under vacuum to obtain the product. A single product is observed by TLC (1:1 MeOH/CHCl₃ as eluent).

Yield=7.07 g, 22.1 mmol, 94.2%.

The purity of the sample was confirmed by ¹H NMR (DMSO-d6, 200 MHz), δ (ppm): NH 9.75, Ar—H (1H) 8.27, Ar—H (2H) 7.94, Ar—H (2H) 7.82, Ar—H (4H) 7.54, CH₂SO₂ 3.09, CH₂CH₂SO₂ 1.69, CH₂ (10H) 1.17, CH₃ 0.83.

Compound IA is characterized by cyclic voltammetry in oxidation and reduction.

The voltammogram (CV) of the compound IA (0.1 M in acetonitrile) in reduction (−0.12 V to +0.90 V vs. Ag/Ag⁺), was measured at 10 mV/s (10 cycles) with a glassy carbon working electrode and a counter electrode with a larger glassy carbon surface area. The reference electrode consists of a silver wire immersed in a solution containing 0.01 M AgNO₃ and 0.1 M tetraethylammonium tetrafluoroborate in acetonitrile.

The voltammogram (CV) of the compound IA (0.1 M in acetonitrile) in oxidation (0.00 V to −3.10 V vs. Ag/Ag⁺) was also measured at 10 mV/s (7 cycles) with a glassy carbon working electrode and a counter electrode with a larger glassy carbon surface area. The reference electrode also consists of a silver wire immersed in a solution containing 0.01 M AgNO₃ and 0.1 M tetraethylammonium tetrafluoroborate in acetonitrile.

FIGS. 1 and 2 respectively show the resulting curves.

Compound I(A) is stable over an electrochemical window of [−2.0 V-0.8 V] vs. Ag/Ag⁺. No significant oxidation waves are apparent (current <50 nA/cm²). A significant reduction current (>1 μA/cm²) can be observed below −2.0 V vs. Ag/Ag⁺.

The lithium salt is then obtained as follows:

Step b)

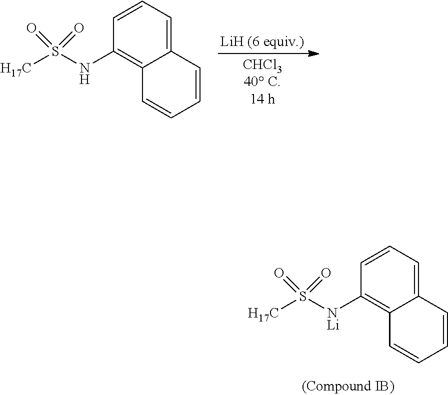

A dry flask is loaded with N-(naphthyl) octanesulfonamide 1 (0.508 g, 1.59 mmol) and CHCl₃ (4 mL) and heated to 40° C. LiH is added slowly (75.8 mg, 9.54 mmol, 6 equivalents) to the stirred solution. The round-bottom flask is sealed and heated with shaking overnight. After 18 hours, MeOH (3 mL) is added dropwise to the reaction mixture. The solution is cooled to room temperature, concentrated under reduced pressure, then diluted with propan-2-ol. The resulting suspension is filtered under reduced pressure and the filtrate collected. Solvents are removed by rotary evaporation.

The resulting compound was analyzed by 1H NMR (see FIG. 3). The disappearance of the sulfonamide proton observed in the starting compound is noted.

Compound IB was characterized by differential scanning calorimetry (DSC) analysis under argon and with a heating rate of 5° C./min from −20° C. to 180° C. The rings are numbered 1, 2, 3.

The results are presented in FIG. 4.

Compound IB has an endotherm at about 20° C. and an exotherm at about 50° C., indicating reversible crystallization/melting over 3 cycles.

### EXAMPLE 2

Synthesis of the Compound IC According to the Invention

Step a)

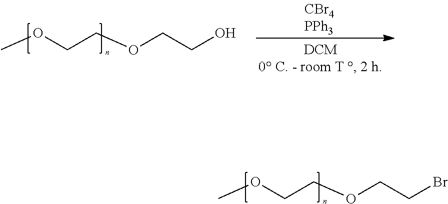

A flask is loaded with a solution of methoxy-PEO-OH (Mn=500 g/mol, 5.00 g, 10 mmol of hydroxy end groups) and CBr₄ (4.97 g, 15 mmol) in dichloromethane (DCM, 25 mL) and cooled to 0° C. To this clear colorless solution is added PPh₃ (3.93 g, 15 mmol) in the form of a dry powder for 15 minutes at 0° C. The solution is allowed to warm up to room temperature and stirring continues overnight. The solution is then concentrated under reduced pressure and precipitated in cold heptane (1×75 mL) and pentane (3×20 mL). The solid is dried under reduced pressure, then taken up in diethyl ether and filtered. Diethyl ether is removed under reduced pressure to obtain the methoxy-PEO-Br product (580 g/mol) in the form of a light-yellow oil (4.26 g, 7.57 mmol, yield 75.7%).

Step b)

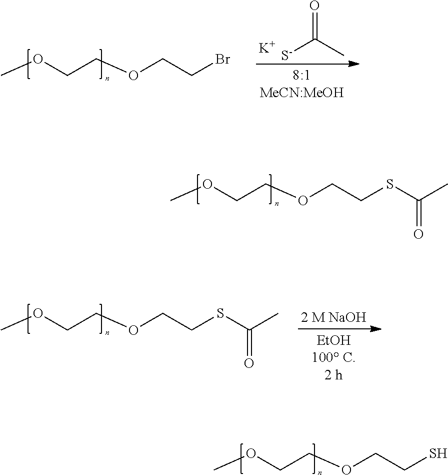

A flask is loaded with methoxy-PEO-Br (580 g/mol, 1.00 g, 1.72 mmol) in MeCN (5 mL). The solution is stirred for 1 hour at room temperature. Potassium thioacetate (295 mg, 2.59 mmol, 1.5 equivalent) is added to the stirred mixture as a solution in MeCN (11 mL)/EtOH (2 mL). The reaction mixture is stirred at room temperature overnight and then concentrated under reduced pressure (109 to 115 mbar, 25 to 28° C.). EtOH (10 mL) is then added, followed by 2 M NaOH (2 mL, 4 mmol). The reaction medium is heated to reflux (100° C., 2 h) and then neutralized by adding 2 M HCl (2 mL, 4 mmol). Volatile compounds are removed under reduced pressure. The oily residue is taken up in DCM and filtered to obtain the crude product of methoxy-PEO-SH in the form of an oil.

Step c)

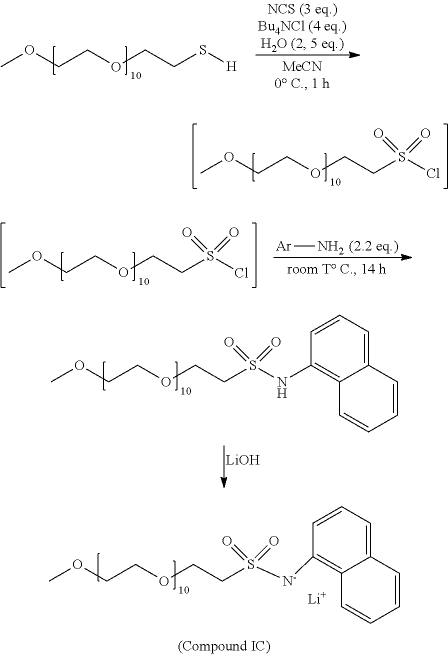

A flask is loaded with methoxy-PEO-SH (500 mg, 516 g/mol, 0.97 mmol) in acetonitrile (MeCN, 10 mL) and deionized water (43.6 mg, 2.42 mmol, 2.5 equivalents) and cooled to 0° C. Tetrabutylammonium chloride (Bu₄NCl, 1.077 g, 3.88 mmol, 4.0 equivalents) and N-chlorosuccinimide (NCS, 388 mg, 29.07 mmol, 3.0 equivalents) are then added and the reaction mixture is stirred at 0° C. for 1 hour. 1-Amino-naphthalene (305 mg, 2.122 mmol, 2.2 equivalents) is then added to the stirred mixture, which is then left at room temperature. The reaction mixture is stirred overnight. MeCN (10 mL) is added to dilute the mixture. The suspension is filtered and the precipitate washed with MeCN (4×10 mL). The solutions are combined and the solvent is removed under reduced pressure. The crude product is taken up in CHCl₃ and passed through a silica gel column. The solvent is evaporated and the residue taken up in deionized water, treated with lithium hydroxide monohydrate up to a pH-metric turn and then extracted with CHCl₃. The combined organic phases are then dried over K₂CO₃ and the solvent removed under reduced pressure to obtain the product in the form of a dark violet oil.

1. An asymmetric sulfonamide compound comprising at least:
a polycyclic group, Ar, consisting of two to six rings, at least one of which is aromatic, said rings each independently having from 4 to 6 members, said polycyclic group being able to include up to 18 heteroatoms;
a linear or branched, saturated or unsaturated aliphatic chain, said chain being optionally interrupted by one or more heteroatoms, by one or more metalloids, optionally substituted by one or more fluorine atoms, and/or containing one or more groups selected from the group consisting of hydroxyl, —NH₂ and oxo groups, said aliphatic chain comprising a linear chain of at least six covalent bonds;
said group Ar and said aliphatic chain being covalently linked via a spacer represented by a sulfonamide unit —SO₂—NH— or its anionic form —SO₂—N⁻—, said sulfonamide being linked to said polycyclic group Ar via its nitrogen atom and to said aliphatic chain via its sulfur atom; and, if need be
a counter cation for the anionic form of the sulfonamide unit, selected from the alkali metals and the proton H⁺ or H₃O⁺.

2. The compound of claim 1, wherein the compound is in the form of a salt having, as spacer, the anionic form of said sulfonamide —SO₂—N⁻—, and a counter cation.

3. The compound of claim 1, wherein the covalent bond between the nitrogen atom and said polycyclic group is established via an aromatic structure forming all or part of said polycyclic group.

4. The compound of claim 1, wherein said polycyclic group Ar is in a condensed aromatic form.

5. The compound of claim 1, wherein said polycyclic group Ar has one of the following polycyclic skeletons or is derived from one of the following polycyclic skeletons:

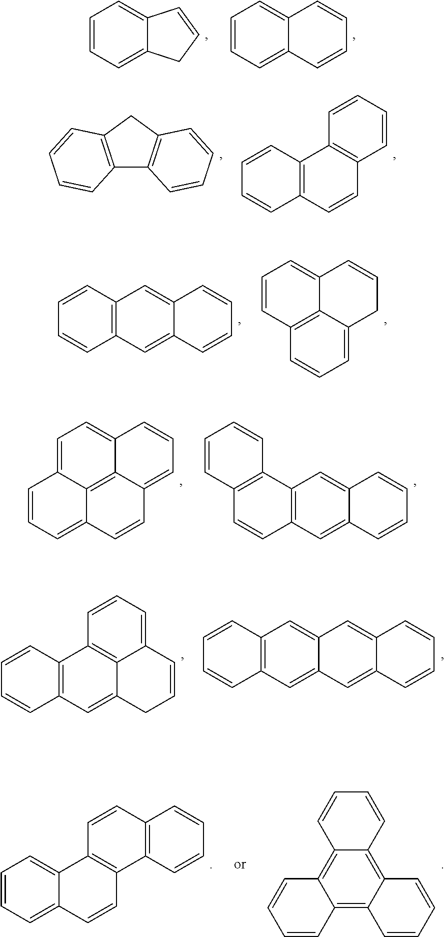

6. The compound of claim 1, wherein said polycyclic group Ar is a radical derived from an aromatic bicyclic skeleton.

7-17. (canceled)

18. The compound of claim 1, wherein said polycyclic group Ar is a naphthalene radical.

19. The compound of claim 1, wherein the aliphatic chain is a linear alkyl chain including from 6 to 18 carbon atoms, optionally fluorinated, semifluorinated, or even perfluorinated, and/or optionally substituted by at least one or more hydroxyl group(s) and optionally intersected by one or more oxygen atom(s).

20. The compound of claim 1, wherein the aliphatic chain has an oligomeric, homopolymeric or copolymeric structure.

21. The compound of claim 1, wherein the aliphatic chain has a chain of at least 5 or more ethylene oxide units.

22. The compound having the general formula: 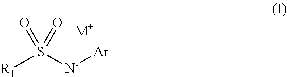
wherein
Ar is a fully aromatic polycyclic group,
R₁ represents an aliphatic chain, and
M⁺ is a counter cation selected from the group of alkali metals, as defined in claim 1.

23. The compound of claim 1 of formula

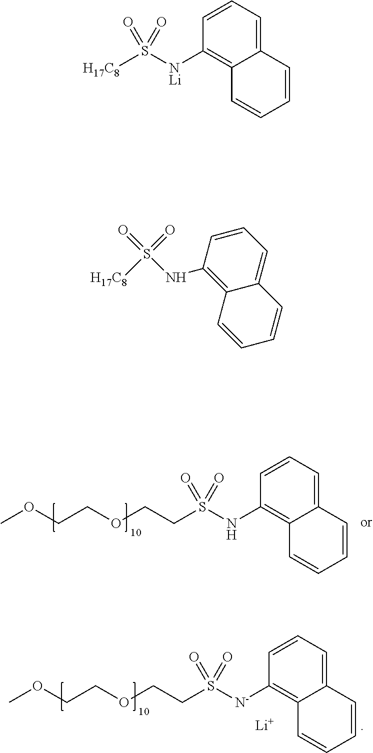

24. A method of preparing an electrolyte, using the anionic form of an asymmetric sulfonamide compound comprising at least:
a polycyclic group, Ar, consisting of two to six rings, at least one of which is aromatic, said rings each independently having from 4 to 6 members, said polycyclic group being able to include up to 18 heteroatoms;
a linear or branched, saturated or unsaturated aliphatic chain, said chain being optionally interrupted by one or more heteroatoms, by one or more metalloids, optionally substituted by one or more fluorine atoms, and/or containing one or more groups selected from the group consisting of hydroxyl, —NH₂ and oxo groups;
said group Ar and said aliphatic chain being covalently linked via a spacer represented by a sulfonamide unit —SO₂—NH— or its anionic form —SO₂—N⁻—; and, if need be
a counter cation for the anionic form of the sulfonamide unit, selected from the alkali metals and the proton H⁺ or H₃O⁺.

25. The method of preparing an electrolyte using the anionic form of the asymmetric sulfonamide compound of claim 1.

26. An electrolyte formed wholly or partially by the anionic form of a compound of claim 1.

27. An electrochemical system comprising an electrolyte formed in whole or in part by the anionic form of an asymmetric sulfonamide compound comprising at least:
a polycyclic group, Ar, consisting of two to six rings, at least one of which is aromatic, said rings each independently having from 4 to 6 members, said polycyclic group being able to include up to 18 heteroatoms;
a linear or branched, saturated or unsaturated aliphatic chain, said chain being optionally interrupted by one or more heteroatoms, by one or more metalloids, optionally substituted by one or more fluorine atoms, and/or containing one or more groups selected from the group consisting of hydroxyl, —NH₂ and oxo groups;
said group Ar and said aliphatic chain being covalently linked via a spacer represented by a sulfonamide unit —SO₂—NH— or its anionic form —SO₂—N⁻—; and, if need be
a counter cation for the anionic form of the sulfonamide unit, selected from the alkali metals and the proton H⁺ or H₃O⁺.

28. An electrochemical system comprising an electrolyte formed in whole or in part by the anionic form of the asymmetric sulfonamide compound of claim 1.

29. The electrochemical system of claim 27, wherein the system is an electrochemical generator, converter or storage system.

30. The electrochemical system of claim 27, wherein the system is a proton exchange membrane fuel cell or redox flow battery, a primary or secondary battery, or a lithium-air or lithium-sulfur battery.
